(19) United States
(12) Patent Application Publication
(10) Pub. No.: US 2019/0048322 A1
(43) Pub. Date: Feb. 14, 2019

(54) METHOD FOR PRODUCING INDUCED PLURIPOTENT STEM CELLS AND INDUCED PLURIPOTENT STEM CELLS PRODUCED THEREBY

(71) Applicant: Chang Hyun KIM, Las Vegas, NV (US)

(72) Inventor: Chang Hyun KIM, Las Vegas, NV (US)

(21) Appl. No.: 15/716,614

(22) Filed: Sep. 27, 2017

(30) Foreign Application Priority Data
Aug. 10, 2017 (KR) 2017-98752

(51) Int. Cl. C12N 5/074; A61B 18/18; A61N 1/40; C12N 5/0735; C12N 5/00

(52) U.S. Cl. CPC C12N 5/0696; A61B 18/18; A61N 1/40; C12N 2506/03; C12N 5/0018; C12N 2506/02; C12N 5/0606

(57) ABSTRACT

According to one aspect of the present invention, there is provided a method for preparing customized pluripotent stem cells and induced pluripotent stem cells thereby. According to this method, a large number of induced pluripotent stem cells can be obtained in high yield. In addition, the induced pluripotent stem cells produced thereby can be used in various fields such as cell therapeutic agents and cosmetic composition.

### CROSS-REFERENCE TO RELATED APPLICATION

This application claims priorities of Korea Patent Application No 2017-98752 filed on Aug. 10, 2017 in the Korea Intellectual Property Office, the entire contents of which are hereby incorporated by reference.

### BACKGROUND OF THE INVENTION

### 1. Field of the Invention

This invention pertains to the method of reprogramming adult stem cells to induced pluripotent stem cells using electromagnetic waves of induced pluripotent stem cells, and to the induced pluripotent stem cells produced thereby.

### 2. Description of the Related Art

There are two main methods for producing customized stem cells. One of them is an embryonic stem cell production method for obtaining stem cells by making somatic cell nuclear transferred embryo, and the other is embryonic stem cell production method where stem cells are obtained by putting various transcription factors into general somatic cells. Embryonic stem cells are cells with pluripotency that have been isolated and cultured from the inner cell mass of the blastocyst, which is the initial stage of fertilization.

In 2006, the method for making induced pluripotent stem cells developed by Dr. Shinya Yamanaka's team at the University of Kyoto was the method where they could generate stem cells from somatic cells by introducing genes through retroviruses used to deliver the four transcription factors of Oct3/4, Sox2, c-Myc and Klf4. (Takahashi and Yamanaka, Cell, Vol. 126, Issue 4, p 663-676, 25 Aug. 2006). However, since retroviral vectors are used in the process of treating transcription factors to normal cells and degeneration of cells may be caused by transcription factors, it is necessary to sufficiently verify whether these cells are safe for use as therapeutic agents.

All the chemical reactions that occur in a cell are caused by electromagnetic vibrations, waves, vibration or the number of vibrations of vibrating atoms, and the elements that make up them. This is the frequency code consisting of all atoms, molecules, cells, and living organisms. All the substances that make up the body actively vibrate at a certain level. If a resonant frequency match occurs in any part of the body, there is a resonance and if these unique codes are identified, then appropriate molecules are absorbed. All cells and cell groups have a resonant frequency embedded in the cell structure during the energy exchange process that takes place on the cell membrane surface. When a natural oscillation speed of a material meets another energy source that vibrates at the same speed, energy transfer occurs, resulting in a biological reaction.

Therefore, it is necessary to develop a technique capable of reprogramming somatic cells or adult stem cells by applying electromagnetic vibrations to somatic cells or adult stem cells.

### SUMMARY OF THE INVENTION

According to one aspect of the invention a sound file storing electromagnetic waves of induced pluripotent stem cells (iPS cells) is transferred to somatic cells or adult stem cells; and There is provided a method to generate customized pluripotent stem cells, including the method to induce pluripotent stem cells by culturing the somatic cells or adult cells.

The term “Pluripotent stem cell” refers to a stem cell with pluripotency that can differentiate into the three germ layers that constitute a living body, that is, the endoderm, mesoderm, and ectoderm. Here, the term “differentiation” refers to a phenomenon where a cell specialize in structure or function when it is proliferating to grow, that is, the process where a cell or tissue of an organism changes in shape or function to perform what they are supposed to do.

The term “induced pluripotent stem cells: iPS cells” is used interchangeably with the term “de-differentiated stem cells” and refers to cells that are induced to produce pluripotency by artificial de-differentiating process (reprogramming) of adult cells that have already undergone differentiation.

The term “customized pluripotent stem cells” refers to the fact that the manufactured pluripotent stem cells are genetically identical to the donated cells (adult cells) used to make the pluripotent stem cells, which means that the customized pluripotent stem cells are originated and induced from the donated cells (adult cells).

The method described above may further include the stage of storing the electromagnetic waves of the iPS cell in a sound file. The step of storing the electromagnetic waves of the iPS cell in a sound file includes the step of placing a culture dish of iPS cells on a spiral coil, the step of flowing a square wave sound signal wavelength into the spiral coil, and the step of storing the electromagnetic waves passed through the iPS cells and the spiral coil in a sound file.

The spiral coil described above may be a planar spiral coil.

The square wave refers to a special wave where a temporal change in voltage or current is a rectangle or a repetitive pattern thereof. The square wave sound signal wavelength may be generated by a square wave generator.

The sound file in the above refers to a file containing information about various sounds or music represented on a computer. The sound file format includes AMP, WAV, VOC, MID, or MP3.

The method described above can induce the S-pole in a culture dish located above the spiral coil by changing the direction of the current flowing through the spiral coil.

The sound file described above may be the one stored in a lossless digital audio recorder. The lossless digital sound recorder is a sound recorder for converting an input analog signal into a digital signal and for storing the digital signal, and the lossless digital sound recorder may record the original sound in a lossless manner.

The above-described method includes the step of delivering a sound file containing electromagnetic waves of an iPS cell to somatic cells or adult stem cells.

The step of delivering a sound file containing electromagnetic waves of the iPS cell to somatic cells or adult stem cells may include: the step of connecting a spiral coil to an output portion of the lossless digital sound recorder storing the sound file, the step of placing a culture dish containing somatic cells or adult stem cells on the spiral coil, and the step of flowing the sound file stored in the lossless digital sound recorder into the spiral coil. The spiral coil described above may be a planar spiral coil.

The above method may induce N-pole into a culture dish located on a spiral coil by changing the direction of the current flowing into the spiral coil.

The above method includes culturing the somatic or adult cells and inducing them to pluripotent stem cells.

The method described above may further include the step of delivering a sound file containing the electromagnetic waves of the iPS cell to somatic cells or adult stem cells, replacing the cell culture with a stem cell culture solution, and culturing the above cell in a replaced stem cell culture solution.

The term “somatic cell” refers to all the cells that make up an animal, excluding the germ cells.

The term “adult stem cell” refers to a cell in an undifferentiated state that can differentiate into cells of a specific tissue when necessary.

The somatic cell or adult stem cell may be a cell derived from a mammal.

The mammal may be the one selected from the group consisting of primates including humans, cows, pigs, bears, cats, dogs, horses, and rodents.

The somatic or adult stem cells may be fibroblasts.

The adult stem cells may be the one selected from the group consisting of adipose derived stem cells, cord blood derived stem cells, bone marrow derived stem cells, neural stem cells, mesenchymal stem cells, and hematopoietic stem cells.

Methods for culturing the cells can use methods known in the art.

The method may be performed without the introduction of a foreign gene, an exogenous chemical, or a combination thereof.

The method described above is characterized as follows: It centrifuges only the supernatant of the medium from which the induced pluripotent stem cells have grown, extracts it, and replace it with the culture medium for a somatic cell or adult stem cell to induce pluripotent stem cells; and then electromagnetic sound signals of the induced pluripotent stem cells recorded in a digital recorder are energized using a planar spiral coil and then it is subcultured. The method may further include culturing the somatic cells or adult cells to induce them to pluripotent stem cells, followed by culturing the cultured cells together with an inactivated feeder cell.

The above feeder cells are, for example, mouse embryonic fibroblasts (MEFs). Another aspect of the invention provides iPS cells prepared according to the method in one aspect of the invention.

The above iPS cells may express at least one from the group consisting of NANOG, OCT4, and SOX2.

Another aspect of the invention provides a cell therapeutic agent including iPS cells according to one aspect of the invention.

The term “cell therapeutic agent”, which is cells and tissues prepared by isolation, cultivation, and special manipulation from a human, is a drug used for therapeutic, diagnostic and prophylactic purposes; it refers to a drug used for therapeutic, diagnostic and prophylactic purposes through a series of actions such as propagating, selecting, or otherwise altering the biological characteristics of allogeneic or xenogeneic cells in vitro to restore the functions of cells or tissues. The above-described cell therapeutic agent may be a stem cell therapeutic agent.

Another aspect of the invention provides a cosmetic composition including iPS cells according to one aspect of the invention. The iPS cells of the cosmetic composition may be from 0.0001 to 5 wt %, 0.001 to 5 wt %, 0.001 to 4 wt %, 0.001 to 3 wt %, 0.001 to 2 wt %, 0.001 to 1 wt %, 0.001 to 0.1 wt % or 0.001 to 0.01 wt %.

The cosmetic composition may contain, in addition to the above extracts, conventional auxiliaries and carriers such as antioxidants, stabilizers, solubilizers, vitamins, pigments or fragrances, and may be prepared into any formulation conventionally produced in the art; for example, solutions, suspensions, emulsions, pastes, gels, creams, lotions, powders, soaps, surfactant-containing cleansers, oils, powder foundations, emulsion foundations, wax foundations and sprays, but is not limited thereto. More specifically, it can be manufactured in the form of flexible lotion, nutritional lotion, nutritional cream, massage cream, essence, eye cream, hair tonic, shampoo, rinse, cleansing cream, cleansing foam, cleansing water, pack, spray or powder.

### BRIEF DESCRIPTION OF THE DRAWINGS

These and other objects, features, aspects, and advantages of the present invention will be more fully described in following detailed description of preferred embodiments and examples, taken in conjunction with the accompanying drawings. In the drawings:

FIG. 1 is a schematic diagram showing a current flow where a magnetic S-pole is formed on the coil as per the Fleming's rule.

FIG. 2 is a schematic diagram showing a current flow where the North Magnetic Pole is formed on the coil as per the Fleming's rule.

FIG. 3 is a schematic diagram showing a current flow where the South Magnetic Pole is formed on a planar spiral coil.

FIG. 4 is a schematic diagram showing a current flow where the North Magnetic Pole is formed on a planar spiral coil.

FIG. 5 is a schematic diagram for storing electromagnetic signals of induced pluripotent stem cells in a sound signal file.

FIG. 6 is a schematic diagram showing a method of flowing an electromagnetic signal stored in the sound signal file of the present invention as a current to a somatic cell dish by using a planar spiral coil.

FIG. 7 is a photo of the induced pluripotent stem cells.

FIG. 8 is a photo of somatic cells (skin cells).

FIG. 9 is a photo of induced pluripotent stem cells transformed from somatic cells.

FIG. 10 is a screen driven by the tone generator software.

FIGS. 11A to 11C are process where one somatic cell is transformed into induced pluripotent stem cells (ips cells).

FIGS. 12A and 128 are photos of a single somatic cell and a collection of somatic cells.

FIGS. 13A to 13E are process where a somatic cell colony is transformed into induced pluripotent stem cells (ips cells).

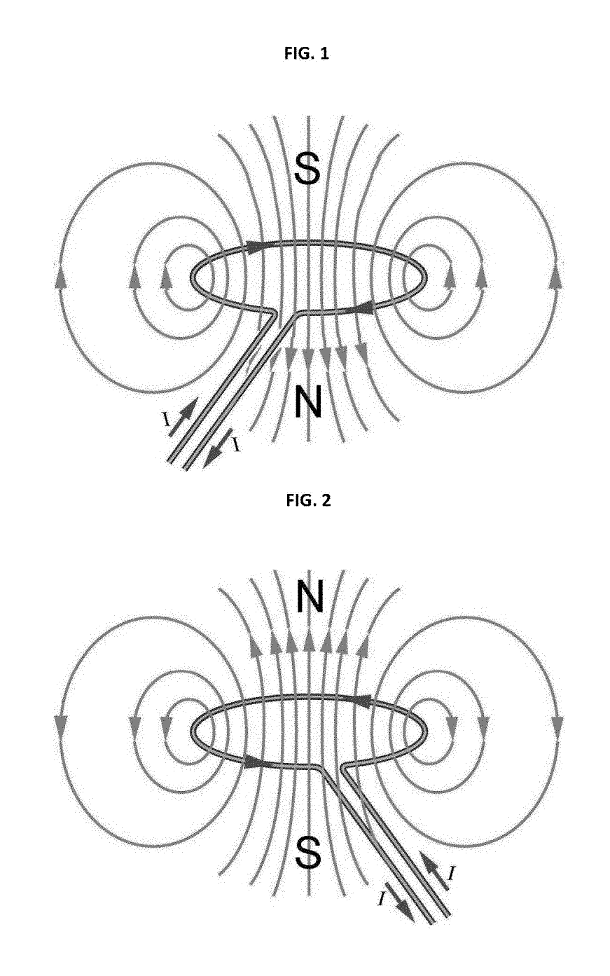

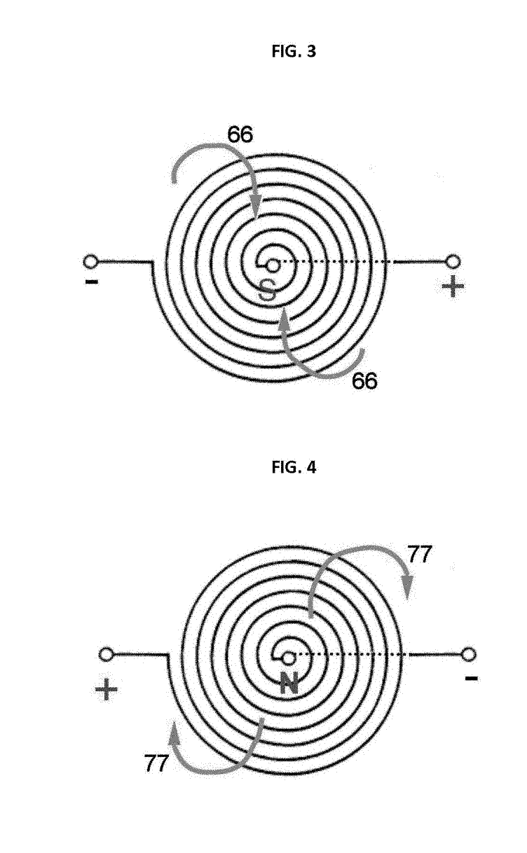

### DETAILED DESCRIPTION OF THE INVENTION

Hereinafter, the present invention will be described in more detail with reference to the Examples. However, these embodiments are for illustrative purposes only, and the scope of the present invention is not limited to these embodiments.

### Example 1

A method for producing induced pluripotent stem cells using electromagnetic waves of induced pluripotent stem cells

### 1. Storage of Electromagnetic Waves of Induced Pluripotent Stem Cells

### 1) Preparation of Samples

(1) Preparation of Culture Media and Culture Dishes

As a culture medium (10) for somatic cell culture, add 10% (v/v) fetal bovine serum (FBS, Gibco BRL Cat No. 16000-044) and 1% antibiotic (Gibco BRL Cat No. 15240-062) to DMEM (Dulbecco's modified Eagles medium, Gibco BRL, Cat No. 11995-065).

As a culture medium for the culture of adult stem cells or induced pluripotent stem cells (iPS cells), knockout DMEM (Gibco BRL, Cat. No. 10829-018) was used together with a 15% (v/v) serum substitute (Gibco BRL Cat. No. 10828-028), 1 mM glutamine, 0.1 mM 3-mercaptoethanol, 1% (w/v) nonessential amino acid (Gibco BRL Cat. No. 11140-050), 4 ng/ml human basic fibroblast growth factor (bFGF) (R&D Systems), 10 ng/ml human oncostatin M (R&D Systems), and 10 ng/ml human stem cell factor (R & D Systems).

Besides, Stemgent Nutristem (XF/FF media Cat. No. 01-0005) was used as a stem cell culture medium (12).

To cultivate adult stem cells without background nutrient cells, a 1:30 mixture of Martrigel and phosphate buffer saline (PBS, Gibco BRL Cat No. 14190-144) and 0.1% (v/v) gelatin, incubated at 4° C. for about 1 hour to coat the dish, and then its supernatant was removed and it was used as a culture dish.

(2) Cryopreservation and Thawing of Somatic Cells or iPS Cells

Somatic cells and iPS cells were prepared, and the cells were cultured with the culture solution described in 1.1). The cultured cells were treated with 3 ml of trypsin and separated, and 7 ml of 0.5% (v/v) PBS was added to the cells and was centrifuged. After centrifugation, the supernatant was removed, and 10 mL of a frozen solution [45 mL of DMEM, 10 g of D-glucose (SIGMA, G-7021), 0.1192 g of HEPES (SIGMA, H-9136), 4 mL of DMSO (SIGMA, D-5879), and 30 mL of FBS was added to the pellet. 1 ml of the cell suspension was dispensed into each of the frozen tubes, stored at −70° C. for about 18 to 24 hours, and stored in a liquid nitrogen tank for freezing.

When frozen-preserved cells were re-cultured, the frozen tubes stored in a liquid nitrogen tank were taken out and thawed in 37° C. water; the thawed cells were washed with DMEM supplemented with 10% (v/v) FBS and then cultured in a 39° C. and 5% CO2, incubator.

(3) Subculture

The somatic cells cultured as depicted in 1.

1) (2) were washed 1-2 times with PBS. 0.05% (v/v) trypsin and 1 mM EDTA solution was added to the washed cells, and the cells remained static for about 5 minutes in the incubator. Thereafter, the cells were washed with PBS, and were recovered. The recovered cells were centrifuged at 1800 rpm for about 5 minutes and washed. The washed cells were dispensed into a new culture dish in an appropriate amount, and DMEM medium supplemented with 10% (v/v) FBS was added to the culture dish and they were cultured in an incubator at about 39° C. and 5% CO2.

2) Placing iPS Cells on Top of a Planar Spiral Coil

To obtain the electromagnetic waves of iPS cells, iPS cells were cultured in BD Matrigel matrix (BD Biosciences, Cat. No. 354602). The BD Matrigel matrix is coated with laminin, collagen IV, heparan sulfate proteoglycan, entactin, and nidogen.

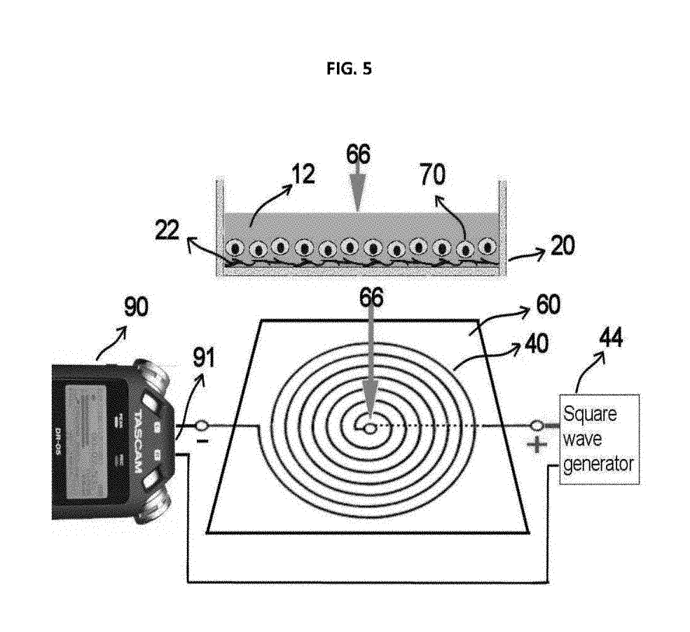

After the cultured iPS cells proliferated to the confluency of about 70% of the culture dish, the iPS cells cultured to store the electromagnetic signals of the iPS cells as lossless files were put on a planar spiral coil (FIG. 5). The planar spiral coil is a coil with an enamel-coated copper wire with a diameter of 0.5 mm wound on a non-conductor plate so as to be spirally arranged around the center and fixed to a flat surface, and it was used in the present invention by connecting the plus and minus terminals of the sound terminal to the electromagnetic signal input terminal 91 at the middle portion and the terminal portion. The planar coil can be made in plate form by etching the copper plate, and in the present invention, it was used with a 0.5 mm copper wire coated with an enamel wound on a circular plate having a diameter of 15 cm around the center in a spiral shape about 10 cm in diameter and with about 6 rotations.

As shown in FIG. 5, the iPS cell culture dish (20) where the iPS cells (70) propagate on the matrigel matrix (22) was placed on the planar spiral coil.

3) Irradiation of Electromagnetic Waves of Electric Square Waves on iPS Cells

As shown in FIG. 5, the iPS cell cultured dish (20) was placed on the planar spiral coil so that the South Magnetic Pole might be formed in the upper layer when the current flowed from the right side of the planar spiral coil (40) to the left side.

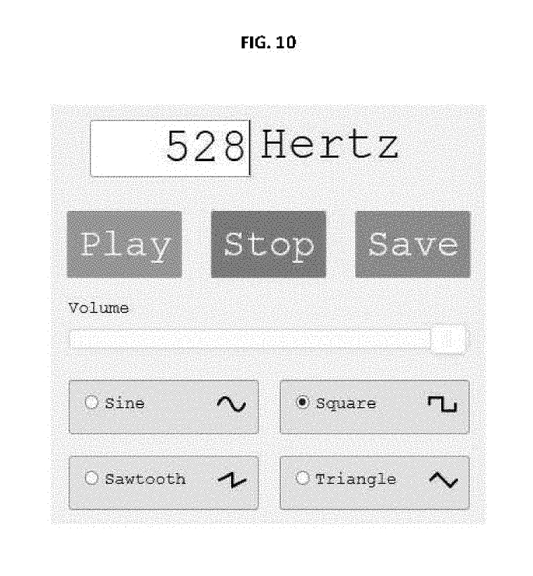

As shown in FIG. 3, a coil is formed in a spiral shape in a clockwise direction from the center of the coil, the thickness of the coil is 0.5 mm, the number of revolutions is 6 rotations, and it is enamelled copper wire. The panel supporting the planar spiral coil consists of an epoxy panel (60). The positive (+) current of a square wave generator (44) is connected to the right side of the planar spiral coil. FIG. 10 shows an execution screen which can generate a square wave sound using a program called a tone generator for an Android phone or a computer, and generates a wavelength of 528 Hz using the program. The square wave generator can generate the sound using the software that generates the square wave and connect the generated sound to the electromagnetic signal input terminal (91) of the recorder to record the sound of the desired frequency, and the recorded sound was used. The square wave generator used the Solfeggio frequencies which could generate the desired frequency sound through the square wave generator software with six sound frequencies of 396 Hz, 417 Hz, 528 Hz, 639 Hz, 741 Hz and 852 Hz, and in the present invention, the 528 Hz sound was recorded and used. The recorded sound can be connected to the positive and negative terminals of the electromagnetic signal output terminal (92) of the WAV sound player to be sent to a planar spiral coil.

4) Storing Electromagnetic Waves of iPS Cells

As shown in FIG. 5, after the sound signal positive (+) current flew from right to left to the underside of the iPS cell culture dish (20), the electromagnetic wave of the sound signal generated in the square wave generator (44) was recorded for about 3 minutes through an input terminal (91) of a lossless sound source recorder (TASCAM DR-05) (90) located on the left side of the planar spiral coil (40). At this time, an S-pole is formed on the planar spiral coil (40).

An electromagnetic sound signal file that was losslessly recorded was named an iPS cell electromagnetic sound file. The format of the iPS cell electromagnetic sound file was WAV file format that is widely used for various purposes.

### 2. De-Differentiation of Somatic or Adult Stem Cells Using Electromagnetic Waves of the Stored Induced Pluripotent Stem Cells

### 1) Connecting a Planar Spiral Coil to the Lossless Digital Recorder Output

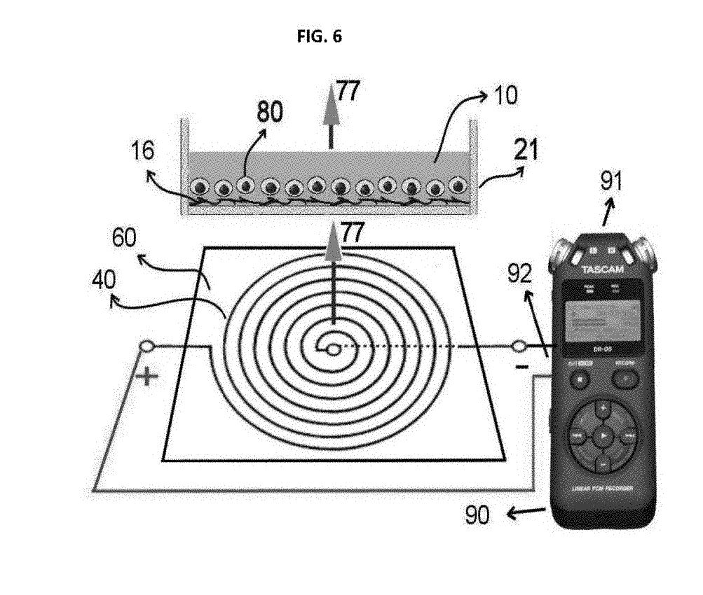

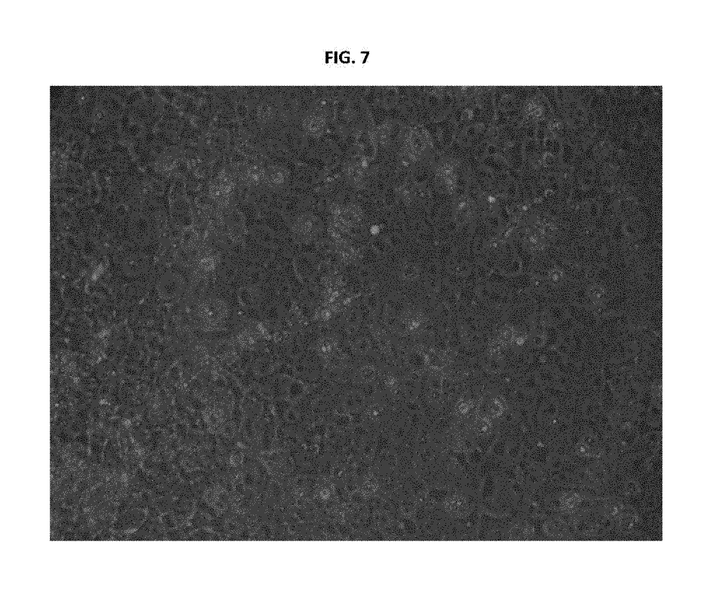

A lossless source recorder (TASCAM DR-05) (90) was connected to the planar spiral coil. As shown in FIG. 6, a (+) plus signal of the sound output terminal (92) of the lossless sound recorder is connected to the center portion of the planar spiral coil (40) and a minus sound terminal is connected to the outer portion of the planar spiral coil for the N-pole to be formed on the planar spiral coil by the induced pluripotent stem cell's electromagnetic sound file signal.

### 2) Arranging Somatic or Adult Stem Cells on Top of the Planar Spiral Coils

Somatic cells or adult stem cells in culture were separated by treating them with 3 ml of trypsin, and after 7 ml of 0.5% (v/v) PBS (Gibco BRL) was added, they were centrifuged. The supernatant was removed, and PBS was added to the pellet to obtain separately isolated cells.

Alternatively, the underside (FIG. 12A) of the culture dish of the somatic cells or adult stem cells in culture was scratched several times with a 1 ml pipet tip to obtain cell aggregates (FIG. 12B).

After the somatic cells or adult stem cells (80) separated individually or prepared as the cell aggregate were put into the culture dish, the somatic cells or adult stem cell culture dish (21) was placed on the upper part of the planar spiral coil (40) as shown in FIG. 6.

The TASCAM DR-05 output terminal 92 is connected and a magnetic line N-pole is formed.

In FIG. 6, the gelatin (16) is coated on the underside of the somatic culture dish, and the somatic cell culture solution (10) used in FIG. 6 was made by collecting only the supernatant after centrifuging the culture solution in which the iPS cells were cultured. The collected supernatant was filtered with a 0.2 μm filter, and about 10 ml to 15 ml of the supernatant was put in a culture dish coated with gelatin (16) together with somatic cells or adult stem cells.

The culture medium in which the existing induced pluripotent stem cells (ips) grew was collected and centrifuged, and then the filtering was used in the present invention; after establishment of the new induced pluripotent stem cell line, only the stem cell culture was used to subculture the new induced pluripotent stem cells.

When the somatic cell culture dish (21) is being placed on the planar spiral coil, the culture medium of the Stemgent Nutristem (XF/FF media Cat #01-0005) where the induced pluripotent stem cells (16) grew by more than 70% of the plate bottom was centrifuged for 4 minutes at 3400 RPM (500 G) centrifuge and filtered, and then only the supernatant is obtained and used as the somatic cell culture medium (10) in the somatic cell culture dish (21) in FIG. 6. In other words, the culture medium where the induced pluripotent stem cells (70) grew is centrifuged and filtered, and then only its supernatant is used as the somatic cell culture medium (10) of FIG. 6.

### 3) Transfer of Electromagnetic Waves of Stored iPS Cells

Using a lossless sound recorder where an iPS cell electromagnetic sound file (Wav) of about 3 minutes in length was stored as depicted in 1.4), while the iPS cell electromagnetic sound file was repeatedly being reproduced with a sound volume of 5 for about 56 minutes, electromagnetic waves was made to flow in a planar spiral coil having a magnetic line N-pole.

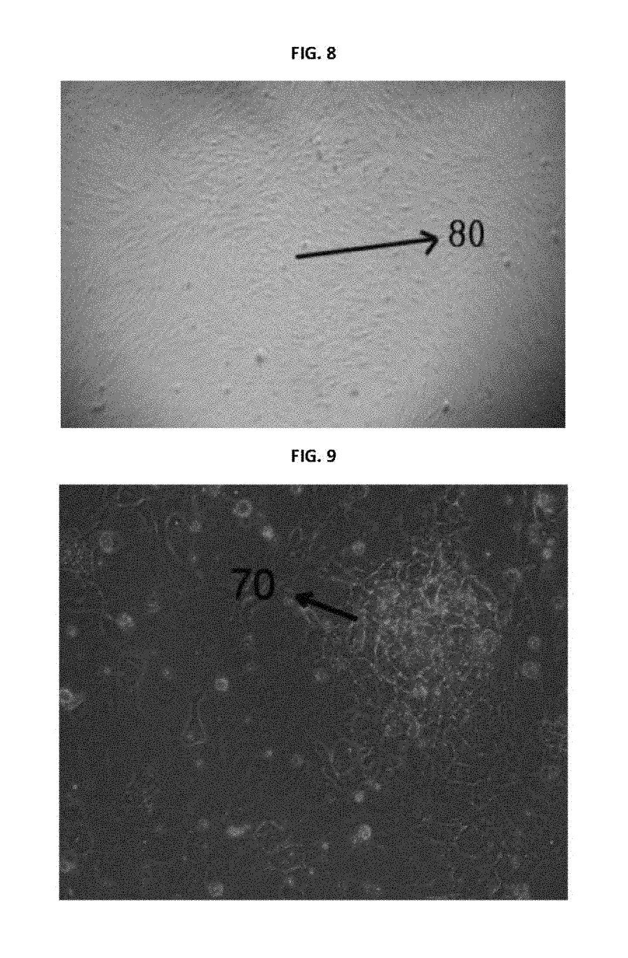

Subsequently, the cell culture was replaced with fresh stem cell culture solution Stemgent Nutristem (XF/FF media, Cat. No. 01-0005), and then the cells were incubated for about 6 to 7 days in an incubator at 37° C. and 5% CO2. By the electromagnetic waves of the stored induced pluripotent stem cells, somatic cell (80) of FIG. 8 was reprogrammed into iPS cell (70) of FIG. 9.

### 4) Characterization of Reprogrammed Cells

As shown in 2.3), cells obtained by transferring the electromagnetic waves of iPS cells to somatic cells or adult stem cells were cultured and examined under a microscope.

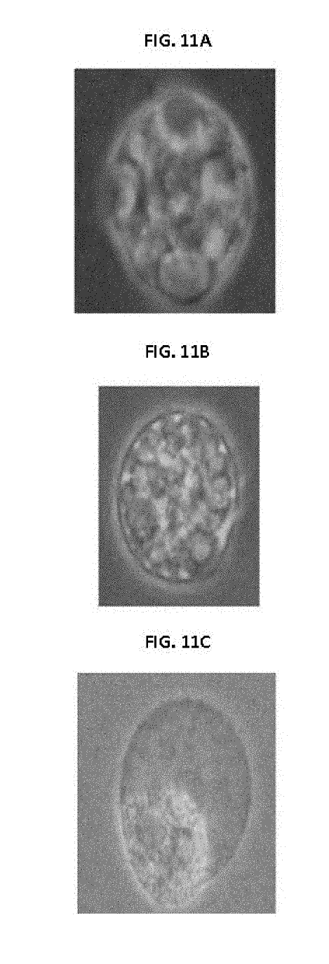

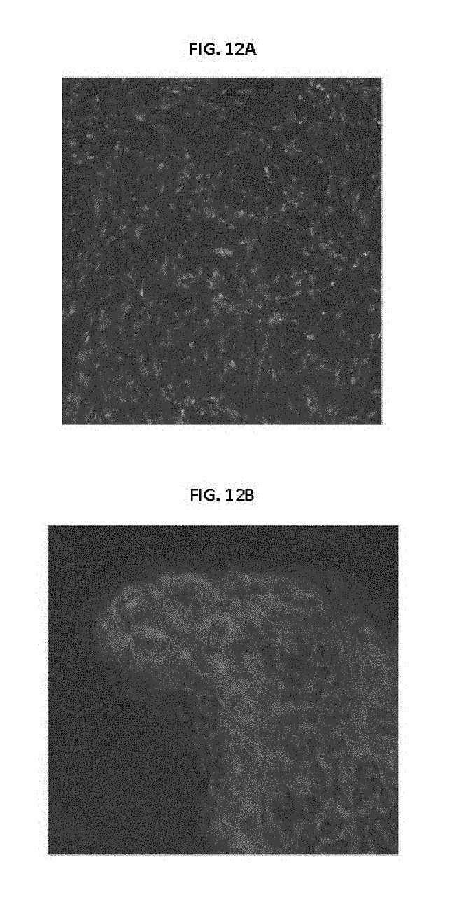

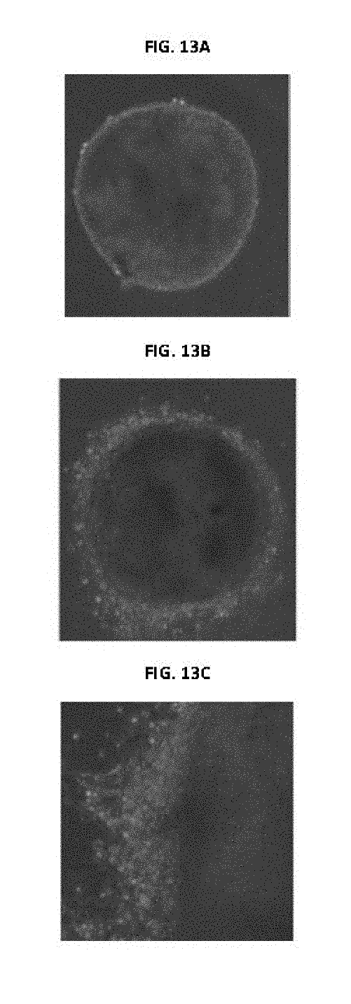

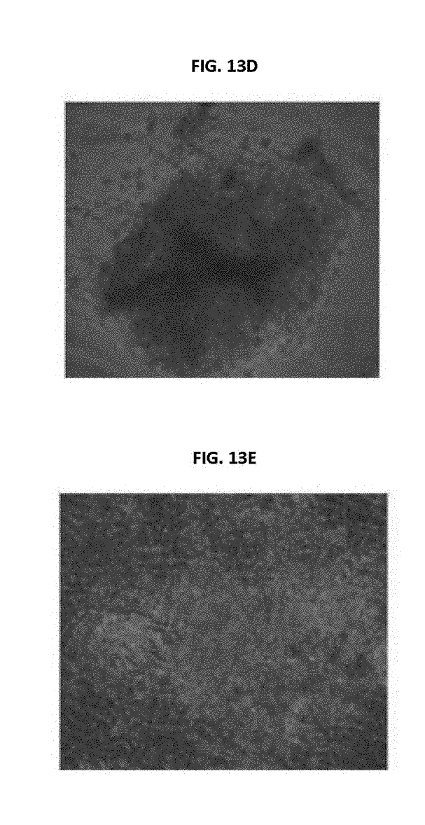

The resultant somatic cells exhibited characteristics of the mammalian embryos after fertilization, even though they were not fertilized (see FIGS. 11A to 11C and 13A to 13E). The size of the isolated cells was smaller than that of the fertilized mammalian embryos, and the zona pellucida was absent. In addition, each cell was divided into blastomeres (FIG. 11A), and all the blastomeres were clustered together and differentiated into morulas (FIG. 11B). Thereafter, expanded blastocysts appeared and then blastocysts (FIG. 11C) appeared. The cell cluster (FIG. 12B) separated from the underside of the plate where a large number of skin cells (FIG. 12A) grew transmitted electromagnetic waves of the iPS cell, blastocoels were generated in the center portion, and then they showed characteristics of blastocysts.

On the other hand, cells obtained by transferring electromagnetic waves of iPS cells to adult stem cells were cultured for about 6 to 7 days in a stem cell culture fluid, and were cultured together with feeder cells for about 10 to 14 days. The cultured cells were confirmed to be iPS cells under a microscope (FIG. 13E). To verify the characteristics of cells obtained by transferring the electromagnetic waves of PSC (Pluripotent Stem Cell), cells were stained with Alkaline phosphatase (Alkaline Phosphatase Live Stain thermofisher code: A14353).

As a result of the staining, the cells were stained purple (FIG. 13D), and the cells obtained by transferring the electromagnetic waves of PSC were confirmed to be iPS cells.

In this way, iPS cell electromagnetic waves were delivered to 5,000 dermal fibroblasts. The culture medium where the ips cells were cultured was centrifuged and filtered with a filter of 0.2 μm; the resultant culture fluid and the mTeSR1 (feeder—free cell culture medium) containing antibiotic cocktail (1% penicillin/streptomycin) was used. The morphology of the cultured cells was confirmed using phase contrast microscopy, and the results are shown in Table 1. As a control group, cells that did not transmit iPS cell electromagnetic waves to skin fibroblasts were used.

TABLE 1

| | | | Shape of cells |
|---|---|---|---|
| | Shape of cells | Shape of cells | look like |
| | look like | look like | expanded |
| | morula stage | blastula stage | blastula stage |
| | (after culture | (after culture | (after culture |
| | 3 day) | 5 day) | 7 day) |
| untreated dermal | 0% | 0% | 0% |
| fibroblasts 5000 | (dermal fibro- | (dermal fibro- | (dermal fibro- |
| cells | blasts condition | blasts condition | blasts condition |
| | continued) | continued) | continued) |
| transmit iPS cell | 80% | 40% | 15% |
| electromagnetic | (4000 cells) | (2000 cells) | (750 cells) |
| waves to dermal | | | |
| fibroblasts 5000 | | | |
| cells | | | |

As shown in Table 1, about 15% of 5,000 dermal fibroblasts were de-differentiated looks like expanded blastula stage shape after 7 days. The inactivated mouse embryonic fibroblasts (MEF) were used as a nutrient cell layer and the expanded blastocyst shape type cells (Not real blastocyst) were established as the induced pluripotent stem cell line after subculture by the general initial stem cell culture method.

While the present invention has been described with reference to preferred embodiments, it will be understood by those skilled in the art that various modifications and variations may be made therein without departing from the scope of the present invention as defined by the appended claims.

What is claimed is:

1. A method for transferring a sound file storing electromagnetic waves of induced pluripotent stem cells (iPS cells) to somatic cells or adult stem cells; and Method of making induced pluripotent stem cells including the induction of cultured somatic cells or adult cells into pluripotent stem cells.

2. The method as claimed in claim 1, wherein the electromagnetic waves of the iPS cell are stored in a lossless sound file.

3. The methods as claimed in claim 2, wherein the electromagnetic waves of the iPS cell is stored as a sound file, wherein a culture dish of iPS cells is placed on a planar spiral coil, wherein a square wave sound signal wavelength flows into the planar spiral coil; and wherein the electromagnetic waves that pass through the iPS cells and the spiral coil are stored in a sound file.

4. The method as claimed in claim 3, wherein the spiral coil is a planar spiral coil.

5. The method as claimed in claim 3, wherein the sound signal wavelength is generated by a square wave generator or tone generator configured as an electrical circuit.

6. The method as claimed in claim 3, wherein an S-pole is induced in a culture dish located above the spiral coil by changing the direction of the current flowing into the planar spiral coil.

7. The method as claimed in claim 3, wherein the sound file is stored as a lossless file in a lossless digital audio recorder.

8. The method as claimed in claim 1, wherein the sound file storing the electromagnetic waves of the iPS cell is delivered to the somatic cells or adult stem cells, wherein a spiral coil is connected to an output portion of the lossless digital sound recorder storing the sound file; wherein a culture dish containing somatic cells or adult stem cells is placed on the spiral coil; and wherein the sound file stored in the lossless digital sound recorder is made to flow into the spiral coil.

9. The method as claimed in claim 8, wherein the spiral coil is a planar spiral coil.

10. The method as claimed in claim 8, wherein the N-pole is induced to a culture dish located above the spiral coil by changing the direction of current flow through the spiral coil.

11. The method as claimed in claim 1, wherein the sound file storing the electromagnetic waves of the iPS cells is delivered to somatic cells or adult stem cells, the cell culture fluid is replaced with a stem cell culture fluid, and the cells are subcultured in the replaced stem cell culture medium until the cells are established as induced pluripotent stem cell lines.

12. The method as claimed in claim 1, wherein the somatic cell or adult stem cells are cells derived from mammals.

13. The method as claimed in claim 1, wherein the adult stem cells are selected from the group consisting of adipose derived stem cells, umbilical cord blood derived stem cells, bone marrow derived stem cells, neural stem cells, mesenchymal stem cells, and hematopoietic stem cells.

14. The method as claimed in claim 1, wherein the method is performed without the introduction of a foreign gene, an exogenous chemical, or a combination thereof.

15. The method as claimed in claim 1, wherein the supernatant of the medium where the pluripotent stem cells are grown is centrifuged and filtered and then replaced with a culture medium of somatic cells or adult stem cells, and then electromagnetic sound signals of induced pluripotent stem cells recorded in a digital recorder were energized using a planar spiral coil and then replaced with a new stem cell culture medium and then subcultured.

16. iPS cells produced by the method according to claim 1.

17. Cell therapeutic agents including the iPS cells according to claim 16.

18. The cosmetic composition including iPS cells according to claim 16.
